(19) United States
(12) Patent Application Publication
(10) Pub. No.: US 2022/0257772 A1
(43) Pub. Date: Aug. 18, 2022

(54) PREPARATION OF LIPOPHILIC ACTIVE INGREDIENTS

(71) Applicant: SPI Pharma, Inc., Wilmington, DE (US)

(72) Inventors: Brian WILSON, Grand Haven, MI (US); Graeme MACLEOD, Wezembeek Oppem (BE)

(73) Assignee: SPI Pharma, Inc., Wilmington, DE (US)

(21) Appl. No.: 17/628,059

(22) PCT Filed: Jul. 17, 2020
(86) PCT No.: PCT/US2020/042680

§ 371 Date: Jan. 18, 2022

Related U.S. Application Data
(60) Provisional application No. 62/876,599, filed on Jul. 19, 2019.

(51) Int. Cl. A61K 47/44; A61K 31/05; A61K 38/13; A61K 47/10; A61K 47/14; A61K 47/26; A61K 9/20; A61P 3/00

(52) U.S. Cl. CPC A61K 47/44; A61K 31/05; A61K 38/13; A61P 3/00; A61K 47/14; A61K 47/26; A61K 9/20; A61K 47/10

(57) ABSTRACT

The present disclosure relates to a formulation for a wide variety of poorly soluble drugs to improve bioavailability using a solid self-emulsifying drug delivery system. Compositions of the present disclosure can be used for improved delivery of hydrophobic or lipophilic pharmaceutical active ingredients, such as drugs, nutritional agents, cosmeceuticals, and diagnostic agents.

### CROSS-REFERENCE TO RELATED APPLICATIONS

This application claims the benefit of U.S. Provisional Application No. 62/876,599 filed on Jul. 19, 2019, which is hereby incorporated by reference in its entirety for all purposes.

### FIELD OF INVENTION

The present disclosure relates to pharmaceutical delivery systems for pharmaceutical active ingredients, such as drugs, nutritionals, cosmeceuticals, and diagnostic agents. More particularly the compositions of the present disclosure relate to the use of lipids and HLB modifying agents to encapsulate or carry one or more active ingredients to prevent First pass metabolism. The present disclosure also relates to a formulation prepared using hot melting process, and/or as part of an additional processing, cooled to a solid lipid particle. The present disclosure also relates to a formulation prepared by placing solid particles into an aqueous liquid to disperse the solid particles into a stable emulsion capable of delivering higher amounts of drug, thereby enhancing dissolution. The present disclosure also relates to compositions comprising a lipophilic active compound, for example a human or veterinary drug or a nutraceutical, with a Hydrophile-Lipophile Balance modifying component.

### BACKGROUND OF THE INVENTION

The therapeutic efficacy of a drug depends upon its bioavailability, which is directly correlated to its solubility. Many drugs, both in development and on the market, are poorly soluble in aqueous media, which can lead to poor bioavailability and frequently results in poor or variable dissolution rates. To achieve the desired drug concentration in systemic circulation to elicit a pharmacological response, solubility is paramount. Different approaches have been taken to achieve a desired level of drug solubility and dissolution rate. These approaches have been based on preparations with increased surface area (micronized powders), molecular inclusion complexes (cyclodextrins and derivatives), co-precipitates with water-soluble polymers (PEG, poloxamers, PVP, HPMC) and non-electrolytes (urea, mannitol, sugars etc.), micellar solutions in surfactant systems (Kolliphor™, Tween™, Gelucires™) and multilayer vesicles (liposomes and niosomes).

Compared to highly soluble compounds, low drug solubility can manifest itself in a variety of unwanted consequences, including (in vivo) decreased bioavailability, an increased chance of a food effect, incomplete release from the dosage form and high inter-patient variability. Despite the formulation challenges, poorly soluble actives are still important class of pharmaceutical compounds for the treatment of a wide range of diseases.

PCT publication PCT/FR2014/00182 discloses self-emulsifying instant powder system with one or a mixture of cyclodextrins for oral administration. US publication 2005/0209345 discloses lymphatic drug delivery system for lipophilic drugs using combination of lipid and surfactant.

U.S. Pat. No. 6,054,136 discloses microemulsion composition comprising an active along with mixture of fatty acid esters and glycerides, surfactant, cosurfactant and hydrophilic phase. EP Patent No. 1058540 discloses immediate release pellet dosage form to improve the bioavailability of an active. The composition disclosed in EP Patent No. 1058540 is based on Self Micro emulsifying Drug Delivery System wherein it comprises a mixture of one or more active ingredients with a lipophilic phase, a surfactant and a cosurfactant.

U.S. Pat. No. 6,572,892 discloses bead composition for dermatological and cosmetology use comprising hydrophobic wax, oil and talcum. U.S. Pat. No. 7,625,507 discloses the extrusion process for forming chemically stable drug multiparticulates which is intended to embrace a dosage form comprising a multiplicity of particles.

US publication No. 2017/0354599 discloses Lipid multiparticulate formulations with a combination of excipients such as a low flow point excipient, and a high flow point excipient. US publication No. 2014/0357708 discloses cannabidiol composition prepared using self-emulsifying system.

Therefore, there is a need for pharmaceutical compositions and dosage forms to attribute the solid system composed of long chain lipids with a surfactant/solubilizing component that is easily processed and solid-stable at 45-65° C. but which when added to gastric media disperses completely with time and forms an emulsion which then enhances drug dissolution and potentially improves BA by absorption via conventional means or alternatively via the lymphatic system.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BRIEF DESCRIPTION OF THE DRAWINGS

The presently disclosed embodiments will be further explained with reference to the attached drawings. The drawings generally illustrate the principles of the presently disclosed embodiments.

FIG. 1 illustrates a dissolution comparison of Fenofibrate loaded at 2.5, 5, and 10% to raw Fenofibrate powder;

FIG. 2 illustrates a dissolution comparison of cannabidiol (CBD) loaded at 2.5, 5, and 10% to raw CBD powder;

FIG. 3 illustrates an example of a liquid triglyceride, or an unsaturated oil;

FIG. 4, comprising FIGS. 4A and 4B, illustrates an example of loading silica with liquid triglyceride, or oil. FIG. 4A is the silica alone (Grace 3150); FIG. 4B is silica with the oil, demonstrating a homogenous mixture;

FIG. 5 illustrates an example of a solid triglyceride (saturated);

FIG. 6, comprising FIGS. 6A, 6B, and 6C, illustrate film casting of fenofibrate active ingredient in solid triglyceride. FIG. 6A is 50 mg/ml fenofibrate in STEROTEX, FIG. 6B is 70 mg/ml fenofibrate in STEROTEX GTP, and FIG. 6C is 50 mg/ml fenofibrate in STEROTEX GTP with 10% TWEEN 80. Film casting is used to determine compatibility of API at different drug loads and the effects of stabilizers, surfactants, and/or cosolvents;

FIG. 7, comprising FIGS. 7A-7D, illustrates compatibility film casting of fenofibrate in solid triglyceride. FIG. 7A is 70 mg/ml fenofibrate in DYNASAN 116, FIG. 7B is 70 mg/ml fenofibrate in STEROTEX GTP (Glyceryl tripalmitate), FIG. 7C is 70 mg/ml fenofibrate in DYNASAN 118, and FIG. 7D is 70 mg/ml fenofibrate in STEROTEX NF (cottonseed oil);

FIG. 8, comprising FIGS. 8A-7D, illustrates compatibility of film casting of fenofibrate in solid triglyceride. FIG. 8A is 70 mg/ml fenofibrate in hydrogenated castor oil (Spectrum), FIG. 8B is 70 mg/ml fenofibrate in STEROTEX K (soybean castor wax), FIG. 8C is 70 mg/ml fenofibrate in STEROTEX HM (soybean oil), and FIG. 8D is 70 mg/ml fenofibrate in cocoa butter;

FIG. 9, comprising FIGS. 9A and 9B, illustrate beads created with DYNASAN 116 (FIG. 9A) and DYNASAN 116 with 50 mg/ml fenofibrate (FIG. 9B) using vibrational drip casting with a Buchi B-390 lab scale spray congealing product.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### SUMMARY OF THE INVENTION

It is an object of the present disclosure to provide an intermediate or a finished formulation of poorly soluble drug. It is a further object of the present disclosure to increase the bioavailability of the formulation of a poorly soluble drug.

The present disclosure also provides a system for the delivery of various active agents which are non-hydrophilic in character within a living body. In another embodiment the present disclosure provides solid lipid particles which forms a solid self-microemulsifying drug delivery system to achieve the increased bioavailability of the poorly soluble drugs.

Thus, in one aspect of the present disclosure, there is provided particles comprising one or more active pharmaceutical ingredient, lipid and/or an HLB modifying agent.

The present disclosure also provides a method of preparing particles comprising one or more active pharmaceutical ingredient, lipid and/or an HLB modifying agent.

Cannabidiol is highly lipophilic, and its oral bioavailability is known to be very low in humans. It is usually supplied via the sublingual route however, it is also available in oil solution, capsule, and nasal spray dosage forms. Cannabidiol is metabolized after administration, and several studies have identified CYP3A4 and CYP2C19 as the major isoforms mediating the metabolism of cannabidiol. Therefore, the oral bioavailability of cannabidiol is affected by both the poor solubility. i.e., low absorption, and the large first-pass effect.

Fenofibrate is hypolipidemic drug used to treat primary hypercholesterolemia, mixed dyslipidemia, and severe hypertriglyceridemia. Fenofibrate is insoluble in water and is rapidly converted to Fenofibric acid (active metabolite) upon oral administration. It is available in the form of tablets and capsules. Like any insoluble drugs. Fenofibrate bioavailability also depends on its solubility.

Fenofibrate is used as a lipid regulating agent and cannabidiol is used for the treatment of epilepsy, anxiety, arthritic pain, sleeping, fibromyalgia, menopause, weight loss, etc. Both of these drugs exhibit poor water solubility and dissolution. Thus, the main object of the present disclosure is to provide the formulation with increased bioavailability for fenofibrate and cannabidiol and drugs with similar physicochemical, pharmacokinetic and pharmacodynamic profiles. The present disclosure also provides the process to prepare fenofibrate and cannabidiol formulation which when placed in the aqueous medium, the solid particles disperse forming a stable emulsion capable of delivering higher amounts of fenofibrate and cannabidiol into solution, thereby enhancing the solubility and consequently the bioavailability of the drug.

The present disclosure also provides the process to prepare fenofibrate and cannabidiol formulations with increased bioavailability.

### DETAILED DESCRIPTION OF THE INVENTION

The present disclosure can provide a number of advantages over conventional methods for the delivery of poorly soluble actives. According to the concept of the present disclosure, the poorly soluble drug is formulated with a lipid and an HLB modifying component.

### Definitions

The term “an active” or “the poorly soluble drug” as used herein interchangeably, refer to an active ingredient that is typically poorly soluble under hydrophilic conditions, including, for example, amiodarone, atorvastatin, azithromycin, carbamazepine, carvedilol, cisapride, cyclosporine, danazol, dapsone, fenofibrate, cannabidiol, gliclazide, glybunde, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezetimibe, cinnarizine, oxyresveratrol, lopinavir, darunavir, olmesartan medoxomil, puararin, lutein, isradipine, lomoxicam, docetaxel, flurbiprofen, ciprofloxacin, furosemide, clopidogrel, dutasteride, amprenavir, saquinavir, calcitriol, valproic acid, isotretinoin, dronabinol, clofazimine, bexarotene, doxecalciferol, sirolimus, dutasteride, tipranavir, paricalcitol, topotecan, loratadine, nintedanib, calcifediol, and mixtures thereof.

The term “lipid” as used herein refers to naturally-occurring, synthetic or semi-synthetic (i.e., modified natural) compound which is generally amphipathic. Lipids typically comprise a hydrophilic component and a hydrophobic component. Exemplary lipids include, for example, fatty acids, fluorinated lipids, neutral fats, phosphatides, oils, glycolipids, surface-active agents (surfactants and fluorosurfactants), aliphatic alcohols, waxes, terpenes, triglycerides, diglycerides, monoglycerides, hydrogenated vegetable oils. Some examples are glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil and mixtures thereof.

The term “monoglycerides” as used herein refers to a molecule derived from glycerol and a single fatty acid, and includes, for example, monolaurin, glyceryl monostearate, glycerol hydroxy stearate and mixtures thereof. Triglycerides are derived from glycerol and three fatty acids. Diglycerides are derived from glycerol and two fatty acids. The term “fatty acid” as used herein refers to a saturated or unsaturated fatty acid, and includes, for example, myristic, palmitic acid, stearic acid, oleic acid and mixtures thereof.

HLB (Hydrophile-Lipophile Balance) is an empirical expression for the relationship of the hydrophilic (“water-loving”) and hydrophobic (“water-hating”) groups of a surfactant. The term HLB modifying component includes, for example, a surfactant, emulsifier, and mixtures thereof.

The term “surfactant” as used herein refers to an amphoteric, non-ionic, cationic, or anionic surfactant and includes, for example, sodium lauryl sulphate, monooleate, monolaurate, monopalmitate, monostearate or another ester of polyoxyethylene sorbitane, sodium dioctylsulfosuccinate (DOSS), lecithin, stearvlic alcohol, cetostearylic alcohol, cholesterol, poly oxyethylene ricin oil, poly oxyethylene fatty acid glycerides, poloxamer®, Kolliphor EL. Kolliphor RH™, Tween™, Gelucires™ and mixtures thereof. Any surfactant is suitable for use in the present disclosure, whether it be amphoteric, non-ionic, cationic or anionic.

The term “emulsifier” as used herein refers to a substance that stabilizes an emulsion, and includes, for example, egg lecithin, soy lecithin, sodium lauryl sulphate and mixtures thereof.

The term “easily processed” as used herein can refer to a system that is a solid, as opposed to a semi solid, and/or a system that it is not sticky or tacky or a system that has reduced stickiness or tackiness.

The term “high melting point” as used herein can refer to a compound or substance having a melting point of at least 30 degrees Celsius (° C.), at least 35° C., at least 40° C., at least 45° C., at least 50° C., at least 55° C., at least 60° C. at least 65° C. at least 70° C., at least 75° C., at least 80° C., at least 85° C., at least 90° C., at least 95° C., at least 100° C. at least 110° C., at least 120° C., at least 130° C., at least 140° C., or at least 150° C.

The term “effective amount” or “therapeutically effective amount” refers to that amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The specific dose will vary depending on the particular compositions chosen, the dosing regimen to be followed, whether the composition is administered in combination with other compositions or compounds, timing of administration, the tissue to which it is administered, and the physical delivery system in which the composition is carried.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “active pharmaceutical ingredient”, “active ingredient”, “single active”, or “API” may refer to an ingredient that is biologically active. In some cases, the pharmaceutical sample contains one API. In some cases, the pharmaceutical sample contains more than one API.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the present disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but can be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of an embodiment of the present disclosure. All compositions, methods, and kits described herein can, in alternate embodiments, be more specifically defined by any of the transitional terms “comprising.” “consisting essentially of,” and “consisting of.”

Overview

The present disclosure provides a composition comprising one or more poorly soluble actives, one or more lipids and/or one or more HLB modifying components. In an embodiment, the amount of poorly soluble active component present in the composition is up to 30% of the total weight of the composition. In an embodiment, the active is present in the composition from about 1% to about 30%, from about 5% to about 30%, from about 10% to about 30%, from about 15% to about 30%, from about 20% to about 30%, or from about 25% to about 30 of the total weight of the composition. In another embodiment, the active is present in the composition from about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of the total weight of the composition.

In an embodiment of the present disclosure, any hydrophobic active ingredient is useful. In another embodiment, the active is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezetimibe, cinnarizine, oxyresveratrol, lopinavir, darunavir, olmesartan medoxomil, puararin, lutein, isradipine, lomoxican, docetaxel, flurbiprofen, ciprofloxacin, furosemide, clopidogrel, dutasteride, amprenavir, saquinavir, calcitriol, valproic acid, isotretinoin, dronabinol, clofazimine, bexarotene, doxecalciferol, sirolimus, dutasteride, tipranavir, paricalcitol, topotecan, loratadine, nintedanib, calcifediol, and mixtures thereof.

In an embodiment of the present disclosure, the active ingredient may be fenofibrate. The active ingredient may be present in an amount of from about 5 mg/ml to about 100 mg/ml, about 10 mg/ml to about 80 mg/ml, about 20 mg/ml to about 75 mg/ml, about 25 mg/ml to about 70 mg/ml, about 30 mg/ml to about 65 mg/ml, about 35 mg/ml to about 60 mg/ml, about 40 mg/ml to about 55 mg/ml, about 45 mg/ml to about 50 mg/ml, about 50 mg/ml, or about 70 mg/ml.

In an embodiment of the present disclosure, the active ingredient is cannabidiol, and is present in an amount of from about 1% to about 10%, from about 5% to about 10%, or at about 5% of the total weight of the final composition.

In another embodiment, the amount of total lipid present in the composition is up to 80% of the total weight of the composition. In an embodiment, more than one lipid is present in the composition. In an embodiment, the total amount of lipid present in the composition is from about 10% to about 80%, from about 15% to about 80%, from about 20% to about 80%, from about 25% to about 80%, from about 30% to about 80%, or from about 35% to about 80%, from about 40% to about 80%, from about 45% to about 80%, from about 50% to about 80%, from about 55% to about 80%, from about 60% to about 80%, from about 65% to about 80%, from about 70% to about 80%, from about 75% to about 80%, or about 80% of the total weight of the composition.

In an embodiment of the present disclosure, the lipid is selected from saturated or unsaturated fatty acids, fluorinated lipids, neutral fats, phosphatides, oils, glycolipids, surface-active agents (surfactants and fluorosurfactants), aliphatic alcohols, waxes, terpenes, triglycerides, diglycerides, monoglycerides, hydrogenated vegetable oils, glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, monolaurin, glyceryl monostearate, glycerol hydroxy stearate, myristic acid, oleic acid, stearic acid, palmitic acid, and mixtures thereof. In an embodiment, the lipid is selected from glyceryl monosterate and glycerol tripalmitate.

In another embodiment of the present disclosure, the HLB modifying component is present in an amount up to 40% of the total weight of the composition. In an embodiment, the total amount of HLB modifying component is present in the composition from about 1% to about 40%, from about 5% to about 40%, from about 10% to about 40%, from about 15% to about 40%, from about 20% to about 40%, or from about 25% to about 40%, from about 30% to about 40%, from about 35% to about 40%, or from about 40% of the total weight of the composition. In an embodiment, the HLB modifying component is selected from one or more of surfactants, emulsifiers, and combinations thereof. In another embodiment, the HLB modifying component is selected from an amphoteric, non-ionic, cationic, or anionic surfactant, sodium lauryl sulphate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer®, Kolliphor EL, Kolliphor RH™, Tween™, Gelucires™, egg lecithin, soy lecithin, sodium lauryl sulphate, and mixtures thereof. In an embodiment, the HLB modifying component is selected from one or more of lecithin and Kolliphor EL.

In an embodiment of the present disclosure, the composition comprises one or more of the lipids glyceryl monostearate (Imwitor 900K) and glycerol tripalmitate (Dynasan 116) and one or more of the HLB modifying components lecithin and Kolliphor EL.

In another embodiment, the present disclosure provides fenofibrate as the active, one or more lipids and/or one or more HLB modifying components. More particularly, the present disclosure provides a fenofibrate composition comprising fenofibrate, glyceryl monostearate (Imwitor 900K), glycerol tripalmitate (Dynasan 116), lecithin and polyethoxylated castor oil (Kolliphor EL).

The present disclosure provides a fenofibrate composition having fenofibrate present up to 30% of the total weight of the composition, glyceryl monostearate (Imwitor 900K) of about 19% of the total weight of the composition, glycerol tripalmitate (Dynasan 116) of about 31% of the total weight of the composition, lecithin of about 10% of the total weight of the composition and polyethoxylated castor oil (Kolliphor EL) of about 10% of the total weight of the composition. In another embodiment, the present disclosure provides fenofibrate composition having an active of about 20% of the total weight of the composition, glyceryl monostearate (Imwitor 900K) of about 22% of the total weight of the composition, glycerol tripalmitate (Dynasan 116) of about 35% of the total weight of the composition, lecithin of about 12% of the total weight of the composition and polyethoxylated castor oil (Kolliphor EL) of about 11% of the total weight of the composition.

In one embodiment, the present disclosure provides cannabidiol as the active, one or more lipids and/or one or more HLB modifying components. More particularly, the present disclosure provides a cannabidiol composition having cannabidiol, glyceryl monostearate (Imwitor 900K), glycerol tripalmitate (Dynasan 116), lecithin and polyethoxylated castor oil (Kolliphor EL).

The present disclosure provides the cannabidiol composition having cannabidiol present up to 30% of the total weight of the composition, glyceryl monostearate (Imwitor 900K) of about 19% of the total weight of the composition, glycerol tripalmitate (Dynasan 116) of about 31% of the total weight of the composition, lecithin of about 10% of the total weight of the composition and polyethoxylated castor oil (Kolliphor EL) of about 10% of the total weight of the composition. In another embodiment, the present disclosure provides cannabidiol composition having an active of about 20% of the total weight of the composition, glyceryl monostearate (Imwitor 900K) of about 22% of the total weight of the composition, glycerol tripalmitate (Dynasan 116) of about 35% of the total weight of the composition, lecithin of about 12% of the total weight of the composition and polyethoxylated castor oil (Kolliphor EL) of about 11% of the total weight of the composition.

The present disclosure also provides the cannabidiol composition having cannabidiol present up to 10% of the total weight of the composition, glyceryl monostearate (Imwitor 900K) of about 25% of the total weight of the composition, glycerol tripalmitate (Dynasan 116) of about 39% of the total weight of the composition, lecithin of about 13% of the total weight of the composition and polyethoxylated castor oil (Kolliphor EL) of about 13% of the total weight of the composition. In another embodiment, the present disclosure provides cannabidiol composition having an active of about 5% of the total weight of the composition, glyceryl monostearate (Imwitor 900K) of about 27% of the total weight of the composition, glycerol tripalmitate (Dynasan 116) of about 41% of the total weight of the composition, lecithin of about 14% of the total weight of the composition and polyethoxylated castor oil (Kolliphor EL) of about 13% of the total weight of the composition.

The present disclosure relates to use of liquid or solid triglycerides in the preparation of an intermediate product comprising a lipophilic active pharmaceutical ingredient (API). Such product may ultimately be used to formulate a dosage form of the API, in particular, an oral dosage form.

In an embodiment of the present disclosure, the intermediate product is comprised of liquid triglycerides and active ingredient, and these components are mixed with silica to create a homogenous mixture. In another embodiment, additional excipients are also included in the mixture to form an oral dosage form such as a sachet or a chewable tablet.

Some examples of liquid triglyceride include sesame oil, olive oil, palm oil, cottonseed oil, corn oil, rapeseed oil, and safflower oil.

Some examples of silica include SYLOID® XDP 3150 (W.R. Grace, Columbia Md.), SYLOID® XDP 3050 (W.R. Grace, Columbia, Md.), ZEOPHARM™ 5191 (Evonik, Parsipanny, N.J.), and ZEOPHARM™ 600 (Evonik, Parsippany, N.J.).

In an embodiment of the present disclosure, solid triglycerides and active ingredient are combined to create a solid form to be spray-congealed into beads. Such beads can then be used to formulate a dosage form, such as an oral dosage form. In one embodiment, the beads and optional additional excipients such as Mannogem® EZ or Pharmasperse®, Pharmaburst® 500 (SPI Pharma, Wilmington, Del.), magnesium stearate, stabilizers, taste masking agents, or any other excipients typical to an oral dosage formulation, are combined to form an oral dosage form, such as a sachet or chewable tablet.

Some examples of solid triglyceride include DYNASAN® 116, DYNASAN® 118 (IOI Olio GmbH, Germany), STEROTEX® GTP, STEROTEX® NF, STEROTEX® K (Abitec, Columbus, Ohio), hydrogenated castor oil (Spectrum Chemical, New Brunswick, N.J.), and cocoa butter.

The present disclosure also provides a process to prepare lipid particle compositions for improving the bioavailability of poorly soluble drugs using a spray coagulation method, film casting method, hot melt extrusion or hot melt granulation method.

Spray Coagulation Method for Creating Solid Lipid Particles

In this method, molten lipid is sprayed into a cooling chamber and on contact with the cool air, congeals into spherical solid particles. The parameters to be considered when preparing a composition according to the present disclosure are the melting point of the excipients, the viscosity of the formulation and the cooling air temperature inside the chamber to allow instant solidification of the droplets. In this application, the spray coagulation method is used to prepare the solid lipid particles. The method is as described below:
a. Glyceryl monostearate (Imwitor 900K), glycerol tripalmitate (Dynasan 116) and polyethoxylated castor oil (Kolliphor EL) are co-melted in 50 ml beaker at 140° C. until the melting process is completed.
b. Once fully melted, lecithin is added to the melt and stirred at 200 rpm.
c. The mixture is then transferred to a heated syringe and allowed to equilibrate to 100° C.
d. The solid lipid mixture is then spray congealed through a Buchi B-390.
e. The mixture is pumped into the heat block, which contains the vibratory atomizer.
f. The nozzle size is set at 80 μm opening and the vibratory atomizer frequency and amplitude are configured to achieve individual droplet separation.
g. Droplets are cooled through a residence time within a cylinder designed to cool ambient air to approximately −6° C.
h. Solid lipid particles are then collected at the bottom of this chamber at a particle size distribution (PSD) between 75-400 μm.

Film Casting Method for Creating Solid Lipid Particles

The Film casting method is a predictive tool used in pre-formulation setting. In this method, a drug-lipid film is cast on glass plates and later milled to a desired particle size. The method used in this application is described as below:
a. Glyceryl monostearate (Imwitor 900K), glycerol tripalmitate (Dynasan 116) and polyethoxylated castor oil (Kolliphor EL) are co-melted in 50 ml beaker at 140° C. until the melting process is completed.
b. Once fully melted, lecithin is then added to the melt and stirred at 200 rpm until the lecithin was fully melted
c. The mixture is then poured onto a room temperature flat surface until the mixture hardened.
d. The solid lipid mixture is then ground until a powder at around 100-300 μm was obtained.

The lipid particles obtained with the processes as described above are further formulated into the final dosage forms such as tablet, sachet, stick packs, capsule, etc., using techniques known in the art. The final dosage form is prepared using inactive excipients like lubricant, glidant, carrier, flavours, sweeteners etc. Lubricants useful in the present disclosure include magnesium stearate, sodium stearyl fumarate, talc, silica, boric acid, sodium benzoate, sodium oleate, sodium acetate, sodium lauryl sulphate, magnesium lauryl sulphate, sodium stearate, magnesium stearate, wax and mixtures thereof. Glidants useful in the present disclosure include magnesium stearate, colloidal silicon dioxide, silica gel, starch, talc and mixtures thereof. Sweeteners used in the present disclosure include sucrose, glucose, glycerol, sucralose, sorbitol, saccharin sodium, aspartame and mixtures thereof. Flavouring agents useful in the present disclosure include orange, chocolate, mint, fruit flavours and mixtures thereof. Carrier systems or diluents useful in the present disclosure include sugar or carbohydrate or polyol-based materials such as mannitol, erythritol, sucrose. Commercial examples of such carrier systems include Mannogem EZ or Pharmasperse 415 and mixtures thereof.

In another embodiment, the tackiness or the stickiness of the lipid particle formulation of the present disclosure can be reduced by adding an additional material to the formulation, by making the lipid particle formulation using a less tacky material, or by increasing the solidity of the material, as described above. It is contemplated that by reducing the tackiness or stickiness of the lipid particle formulation, the resultant particles can be more stable when packaged together (e.g., reducing aggregation of the particles in the package) or stored together. This concept is further expanded upon below (see, e.g., Suspension of solid).

Particle Coating

Once particles are created through either method described above, particles can be coated with titanium dioxide, silicon dioxide, mannitol, lactose, calcium silicate, magnesium stearate, and starch to reduce particle to particle connections that cause agglomeration.

Suspension of a Solid

A material with a high melting point can be added to the lipid mixture once the lipid mixture prepared above is melted (e.g., to reduce or eliminate the tackiness or stickiness of the lipid particle formulation). In certain embodiments, about 5% to about 50% by weight of a material with a high melting point can be added to the lipid mixture. In certain embodiments, about 5%, about 10%, about 15%, about 20%, about 25%, about 30%, about 35%, about 40%, about 45%, or about 50% by weight of a material with a high melting point can be added to the lipid mixture. The material added is required to remain solid, where the addition of a soluble or insoluble material can significantly increase the viscosity of the material to further solidify the material. The addition of a solid material can also act as pore former or swelling agents to increase disintegration time of the solid lipid particles where the solid material is also soluble. Examples of pore formers or swelling agents include materials such as sugars (e.g. sucrose, glucose, maltose), polyols (e.g. mannitol, maltitol, lactitol), and hydrophilic polymers (e.g. PVP, PVA, HPMC, PEG 400-8000, sodium starch glycolate, or 1-HPC).

Co-Melting with a Polymer

Polymers with moderate to high melting points like PEG 1500-8000, Poly vinyl alcohol, and Polox can be co-melted with the lipid system to add stability to the particles and also act to increase the wetting or disintegration time of the particles.

In one embodiment, the lipid particles prepared using spray coagulation method or film casting method can be further coated to improve their processability or functionality. The coating of the lipid particles can be done using the method as described below:

500 g of the lipid particles can be loaded into a lab scale fluid bed processor such as a GPCG 1.1 Glatt coater using a Wurster column and bottom spraying approach. A solution of HPMC can be prepared by dissolving the HPMC E15 grade in water at around 10% weight loading. The resultant solution can be applied to the pellets by fluidizing them in air and applying the coating via the nozzle and atomizing the solution. The product temperature during the coating operation would be around 40° C. An alternative coating material to apply to the multiparticulates could be ethylcellulose which comes as a latex suspension in the form of Aquacoat or Surelease. The Aquacoat would be plasticized via the addition of 22% (calculated as a % of the solids in the dispersion) of a plasticizer such as triethyl citrate stirred under low agitation for around 30 mins. The Aquacoat or Surelease systems could be diluted by the addition of water prior to use by adding up to 40% w/w water. The same process approach could be undertaken to apply the ethylcellulose based systems. The total polymer weight loading applied would be around 3-10% expressed as a % of the multiparticulate weight and depending on the polymer system used. Other polymers that could be applied to the multiparticulates include polymethacrylates, cellulose acetates, hydroxypropyl methylcellulose phthalate, HPMCAS (hydroxypropyl-methylcellulose acetate succinate), polyvinyl alcohol. Commercial examples of these types of polymers that are preformulated for ease of use include Opadry. Eudragit, Kollicoat and Methocel. Polymer coatings could also be applied by using powder layering techniques.

The present disclosure is further explained with the following examples. The example is demonstrated by using cannabidiol (CBD) as an active wherein solid lipid particles of CBD are prepared and later used in preparing a final dosage form e. g. sachet, stick pack, orally dispersible powder, orally dispersible tablet, lozenge, chewable or swallow tablets.

### EXAMPLES

### Example 1—Preparation of Solid Lipid Particles of Cannabidiol

| | Percentage of |
|---|---|
| Component | formulation |
| Imwitor 900K | 27% |
| Dynasan 116 | 41% |
| Lecithin | 14% |
| Kolliphor EL | 13% |
| CBD | 5% |
| Total | 100% |

Detailed Method of Preparation:

Method 1—Spray Coagulation Method for Creating Solid Lipid Particles of Cannabidiol

Imwitor 900K, Dynasan 116, and Kolliphor EL were co-melted in a 50 ml beaker at 140° C. Once fully melted, lecithin was added to the melt and stirred at 200 RPM until lecithin completely melted. Cannabidiol (CBD) was then added and co-melted in the mixture. The mixture was further transferred to a heated syringe and allowed to equilibrate to 100° C. The solid lipid mixture was then spray coagulated through a Buchi B-390 and the mixture was pumped into the heat block having the vibratory atomizer.

The nozzle size was set at 80 μm opening and the vibratory atomizer frequency and amplitude were configured to achieve individual droplet separation Droplets were further cooled through a residence time within a cylinder designed to cool ambient are to ˜−6° C. Solid lipid particles were then collected at the bottom of this chamber at a PSD between 150-200 μm.

Method 2—Film Casting Method for Creating Solid Lipid Particles of Cannabidiol

Imwitor 900K, Dynasan 116, and Kolliphor EL were co-melted in a 50 ml beaker at 140° C. Once fully melted, lecithin was added to the melt and stirred at 200 RPM until lecithin until lecithin completely melted. Cannabidiol (CBD) was then added and co-melted in the mixture. The mixture was then poured onto a flat surface at room temperature and was allowed to cool until hardened. The solid lipid mixture was then ground until a powder at around 100-300 μm obtained.

Solid lipid pellets derived through either spray coagulation or film casting can be used in orally dispersible powder formulations or in tablets prepared using direct compression process.

Sachet and Stickpacks of Cannabidiol

A. Using Solid Lipid Particles Only

90-98% of Solid lipid pellets comprising an active are mixed with magnesium stearate at a level of about 1-5% for 5 minutes. 0.25-1% of fruit flavour and 0.1-1.5% of sucralose are further added to the mixture and the mixture is filled into an aluminium sachet using manual filling or an automated stick pack filling machine and packed as sachets or as stick packs.

The sachet or a stick pack prepared according to a method of the present disclosure is further explained with the following example:

| Component | Percentage |
|---|---|
| Solid lipid pellet of CBD | 96.75% |
| Magnesium stearate | 2% |
| Fruit flavour | 0.75% |
| Sucralose | 0.5% |
| Total | 100% |

B. Using Solid Lipid Particles with a Carrier

Mannogem EZ (a spray dried mannitol) or other carriers like Pharmasperse 415 can be added to assist as a flow agent and carrier. About 5-40% of the soluble carrier system is added to 56.75-92.25% of the solid lipid particles and mixed for 5-15 minutes. Magnesium stearate of about 2% to act as a glidant is added to the mixture for 5-10 minutes. 0.25-1% of fruit flavour and 0.1-1.5% of sucralose are further added to the mixture. The mixture is then filled into an aluminium sachet using manual filling or an automated stick pack filling machine and added by weight to the desired dosage.

The sachet or a stick pack prepared with carrier system according a method of the present disclosure is further explained with the following example:

| Component | Percentage |
|---|---|
| Solid lipid pellet of CBD | 72.5% |
| Garner (Mannogem EZ or | 24.25% |
| Pharmasperse 415) | |
| Magnesium stearate | 2% |
| Fruit flavour | 0.75% |
| Sucralose | 0.5% |
| Total | 100% |

Tablets of Cannabidiol

0.1-80% of the Solid lipid particles of CBD of about are blended with 20-75% of diluent and 0.1-3% of lubricant. The obtained mixture is then compressed into a tablet.

The tablet prepared according a method of the present disclosure is further explained with the following example:

| Component | Percentage |
|---|---|
| Solid lipid particles of CBD | 75% |
| Diluent [Mannitol (Mannogem XL)] | 24.75% |
| Lubricant (Mg stearate or sodium stearyl | 0.25% |
| fumarate) | |
| Total | 100% |

The following reference examples are further offered to illustrate, but not to limit, the present disclosure to embodiments provided in the reference examples.

### Reference Example 1

| | Percentage of |
|---|---|
| Component | formulation |
| Imwitor 900K | 27.8% |
| Dynasan 116 | 40.7% |
| Lecithin | 13.1% |
| Kolliphor EL | 12.4% |
| Progesterone | 6.0% |
| Total | 100% |

### Reference Example 2

| | Percentage of |
|---|---|
| Component | formulation |
| Imwitor 900K | 25.8% |
| Dynasan 116 | 38.8% |
| Lecithin | 13.0% |
| Kolliphor EL | 12.4% |
| Docusate | 5.0% |
| Cyclosporin | 5.0% |
| Total | 100% |

### Reference Example 3

| | Percentage of |
|---|---|
| Component | formulation |
| Imwitor 900K | 25.8% |
| Dynasan 116 | 38.8% |
| Lecithin | 13.0% |
| Kolliphor EL | 12.4% |
| Furosemide | 10.0% |
| Total | 100% |

### Reference Example 4

| | Percentage of |
|---|---|
| Component | formulation |
| Imwitor 900K | 25.8% |
| Dynasan 116 | 38.8% |
| Lecithin | 13.0% |
| Kolliphor EL | 12.4% |
| Docusate | 5.0% |
| Desmopressin | 5.0% |
| Total | 100% |

In another embodiment, the present disclosure also teaches preferred silica formulation used in a formulations of the present disclosure.

Docusate and Propyl Gallate

The use of 12% docusate showed the fastest release of CBD with a low change in the release profile over 1 month at 40° C. 75% RH. As known, docusate is not commonly used in these formulations. Therefore, the findings show that docusate or ionic surfactants are strong candidates for immediate release in silica formulations.

| | Percentage of |
|---|---|
| Component | formulation |
| Sesame Oil | 32% |
| CBD | 9% |
| Syloid 3150 | 46% |
| Docusate | 12% |
| Propyl gallate | 1% |
| Total | 100% |

PEG and Propyl Gallate

The use of 12% PEG 400 showed a moderate release of CBD with a very low change in the release profile over 1 month at 40° C., 75% RH.

| | Percentage of |
|---|---|
| Component | formulation |
| Sesame Oil | 32% |
| CBD | 9% |
| Syloid 3150 | 46% |
| PEG 400 | 12% |
| Propyl gallate | 1% |
| Total | 100% |

Docusate+Kolliphor EL+Propyl Gallate

The use of 6% Kolliphor EL and 6% Docusate showed a moderate release of (CBD with the lowest change in the release profile over 1 month at 40° C., 75%, RH.

| | Percentage of |
|---|---|
| Component | formulation |
| Sesame Oil | 32% |
| CBD | 9% |
| Syloid 3150 | 46% |
| Kolliphor EL | 6% |
| Docusate | 6% |
| Propyl gallate | 1% |
| Total | 100% |

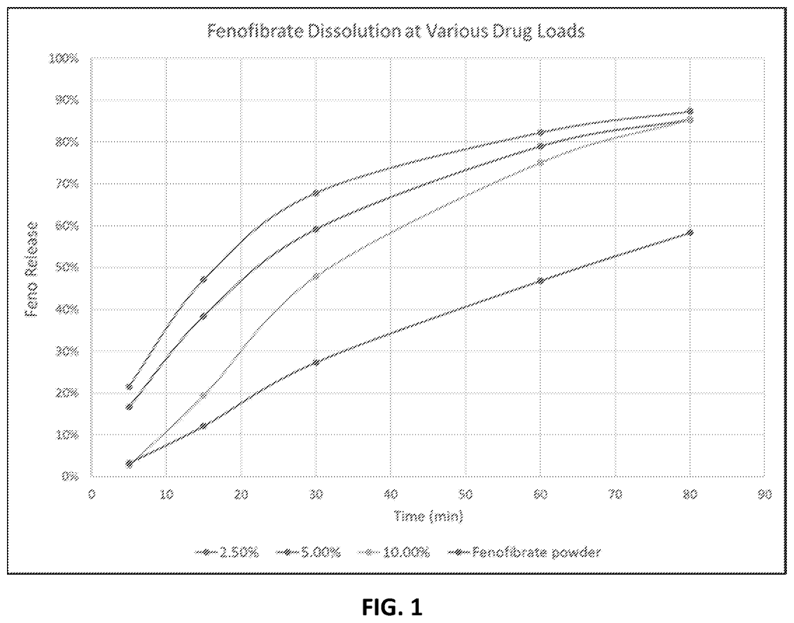

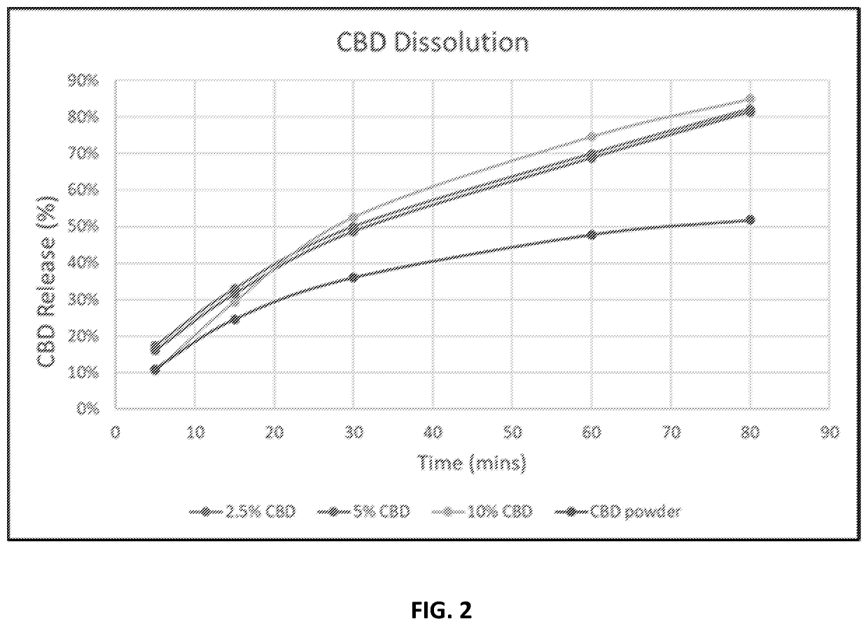

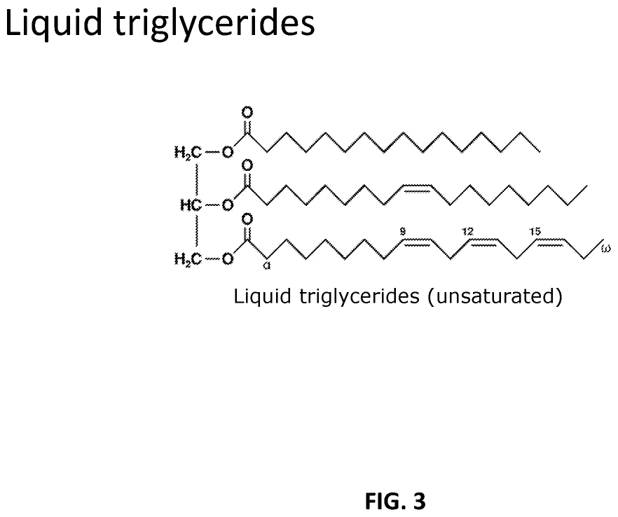

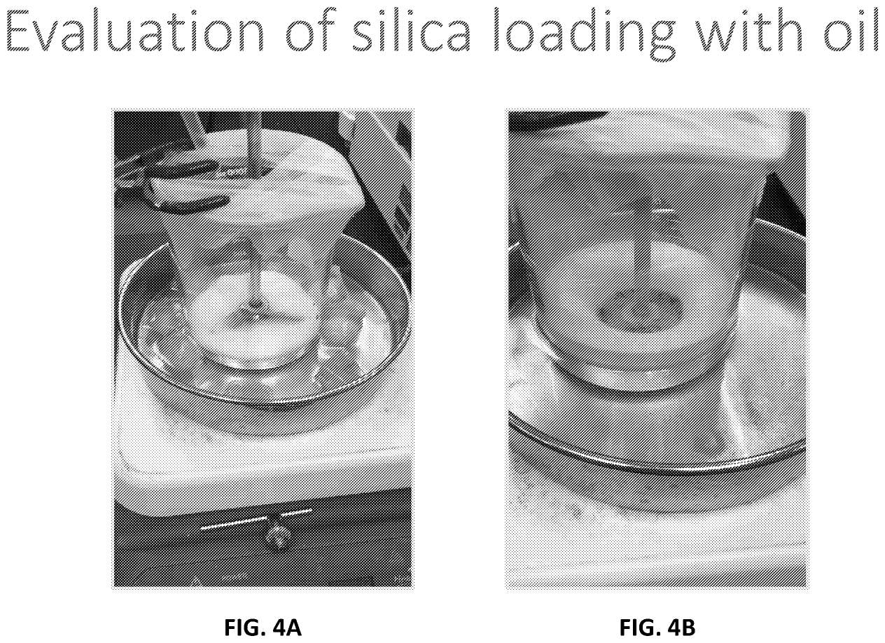

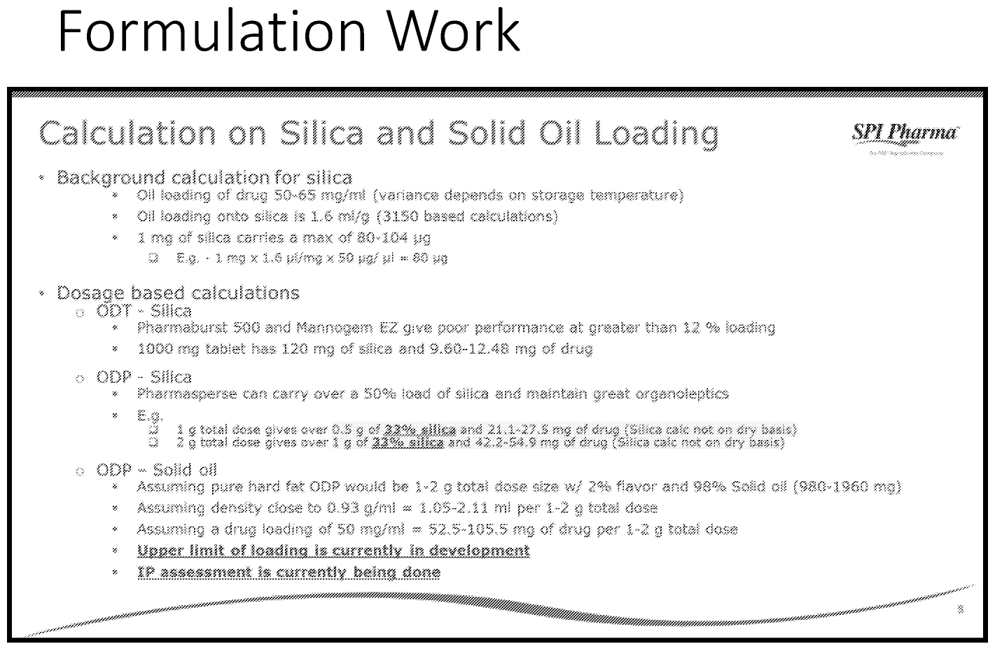

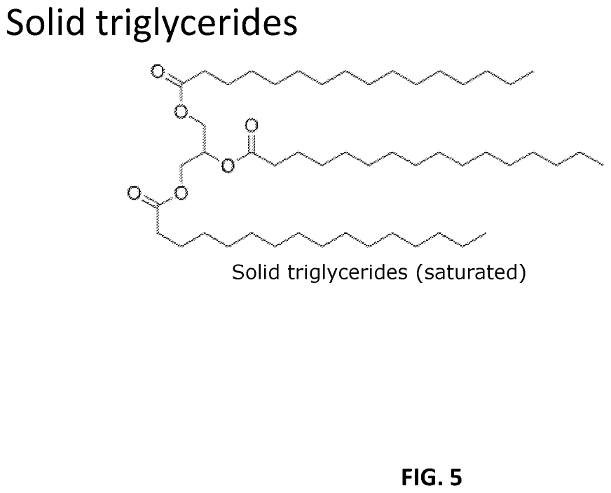

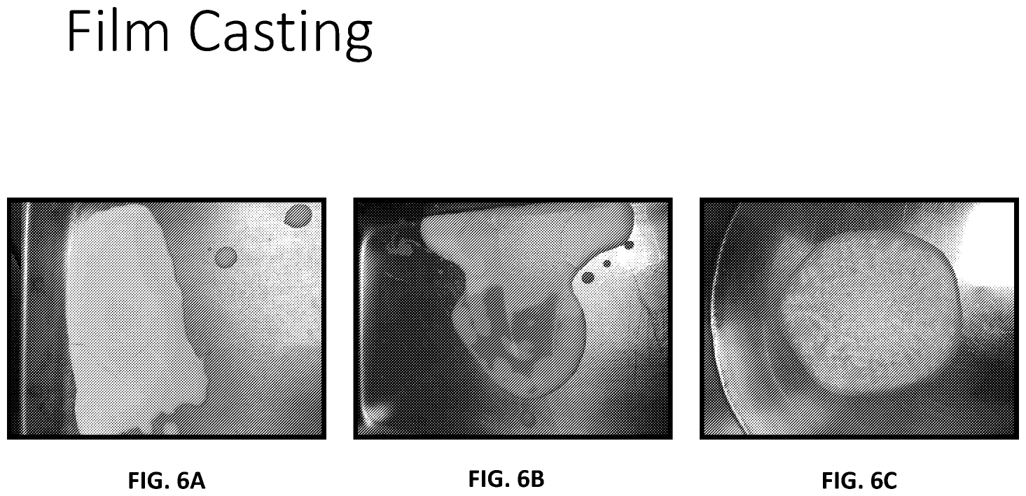

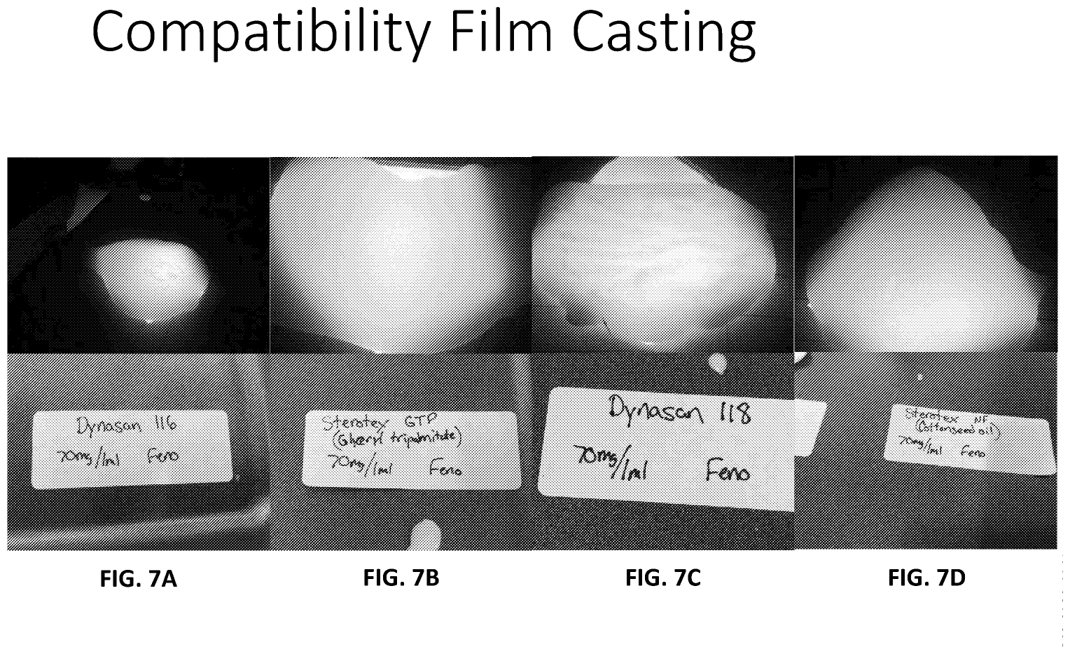

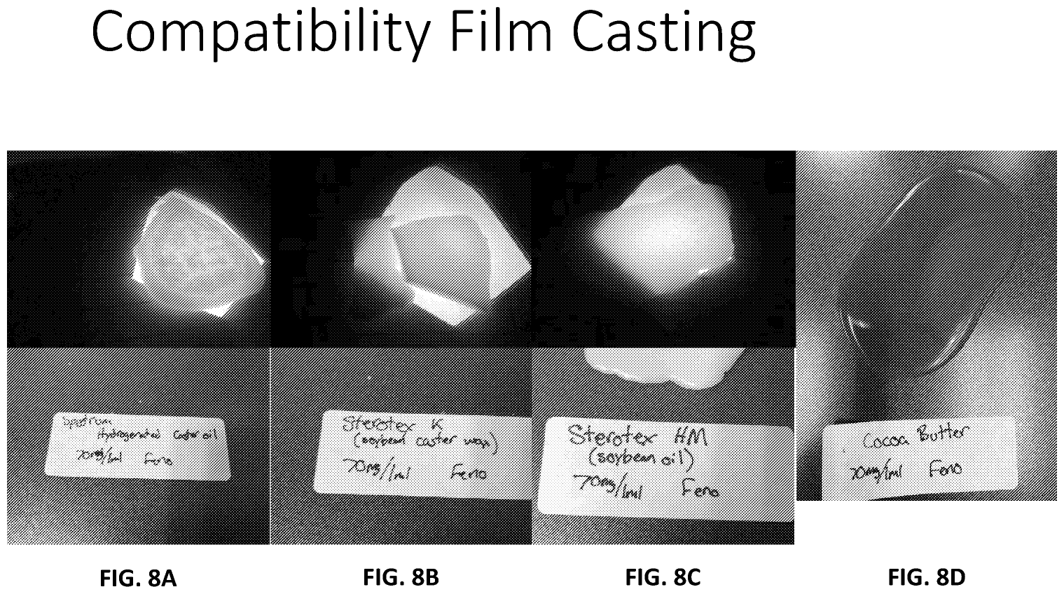

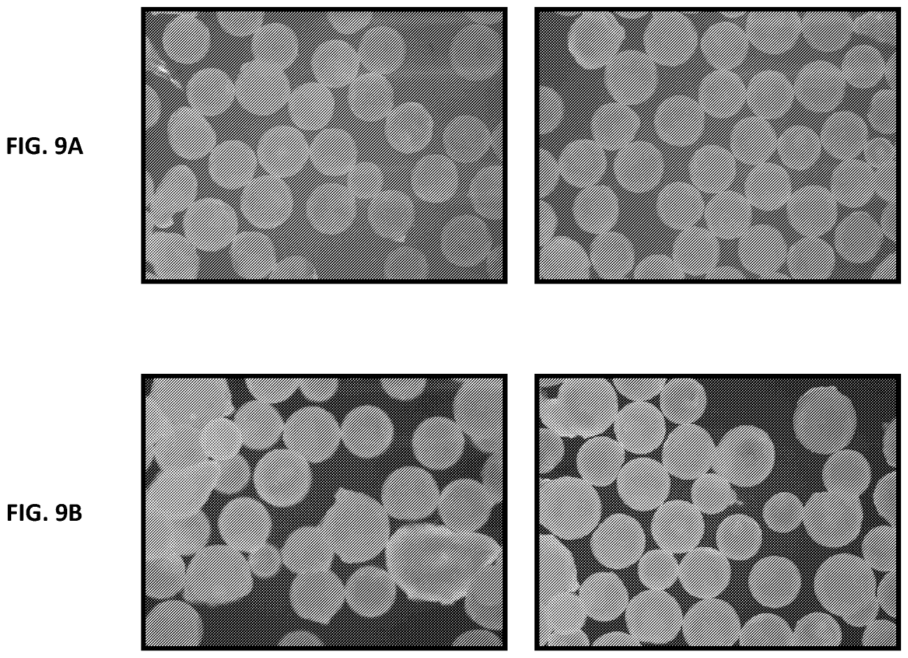

In one embodiment, the present disclosure further demonstrates the increased bioavailability of the formulation of the present disclosure with the dissolution profile as shown in FIGS. 1 and 2. The formulation of the present disclosure (Fenofibrate formulation) is compared with the placebo formulation.

Dissolution Media Conditions

The dissolution media is a 6.8 Phosphate buffer with 2% Tween 80. The media used to observe both cannabidiol and fenofibrate release using the USP Apparatus 2, 1 L per vessel, 37° C., and at 50 rpm. The solubility of actives studied was shown to be above amounts within this dissolution media.

Emulsion Formation and Dissolution Profile:

The formulation disintegration was tested using a placebo formulation or Fenofibrate at 5, 7.5, and 10%. Each mixture was placed into dissolution buffer mixed at 150 RPM and a temperature of 37° C. Each mixture was observed to have fully dispersed into the dissolution buffer between 10-15 minutes to form emulsions. After 60 minutes, the mixture can be poured through a 20 μm screen, leaving no visible residue.

FIGS. 1 and 2 further compare the dissolution profile of fenofibrate and cannabidiol (CBD) formulation, respectively, prepared according to the present disclosure vis-b-vis the raw powder of fenofibrate and cannabidiol.

Stability Data:

The pellets of example 1 were maintained in open conditions and exposed to a temperature of 23° C. and 30% RH for 30 days. There were no detectable impurities related to oxidation or photo degradation when analysed using a UPLC method.

### Example 2—Loading of Silica with Oil

Silica loading depends on mixing energy, rate of oil addition, droplet size, and temperature. Silica loading using SYLOID® XDP 3150 was found to cap out at 1.6 mg/ml. Pilot testing for optimal oil loading for ZEOPHARM 5191, ZEOPHARM 600 and SYLOID® XDP 3150 demonstrated results of about 2 mg/ml, 3 mg/ml, and 1.4 mg/ml, respectively.

### Example 3—Formulating the Silica/Oil/API Intermediate into an Oral Dosage Form

API is loaded into oil at 50-65 mg/ml, depending on a variety of factors including storage temperature. Oil loading onto silica is about 1.6 ml/g using SYLOID® XDP 3150. One mg of silica carries a maximum load of about 80-104 μg of API. For an orally dissolving tablet (ODT) using the oil/silica/API intermediate product, PHARMABURST® 500 or Mannogem EZ may be used to formulate the ODT at not greater than 12% silica (e.g., a 1000 mg tablet has 120 mg silica and 9.6-12.48 mg of API). Based on this information, greater than 50% loading is likely possible.

To formulate an orally dispersible platform (ODP), PHARMASPERSE® may be used in combination with the oil/silica/API intermediate product. PHARMASPERSE® can carry a load of 50% or more of the silica intermediate and still maintain excellent organoleptics. A 1 g dose can deliver 0.5 g of 33% silica and 21.1-27.5 mg of drug. A 2 g dose can deliver 1 g of 33% silica and 42.2-54.9 mg of drug. Other excipients, such as magnesium stearate, may be used in the ODP formulation.

### Example 4—Formulating the Solid Triglycende/API Spray-Congealed Beads into an Oral Dosage Form

For a 1 to 2 g total dose size, the oral dosage form may comprise about 2% flavor and about 98% solid oil. A density of about 1.05-2.11 ml per 1-2 g dose, and drug loading of at least about 52.5-105.5 mg per 1-2 g dose, are expected.

1. A lipid particle composition, comprising:
one or more poorly soluble active ingredients;
one or more lipids; and/or
one or more Hydrophile-Lipophile Balance (HLB) modifying agents to improve a bioavailability of the one or more poorly soluble active ingredients within a subject.

2. The lipid particle composition of claim 1, wherein the one or more lipids and/or the one or more HLB modifying agents comprise a solid system, and wherein the solid system is easily processed and/or stable at a temperature ranging from between about 45° C. and about 65° C.

3. The lipid particle composition of claim 2, wherein the one or more lipids comprise long chain lipids.

4. The lipid particle composition of any one of claims 1-3, wherein the one or more poorly soluble active ingredients is selected from the group consisting of amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezetimibe, cinnarizine, oxyresveratrol, lopinavir, darunavir, olmesartan medoxomil, puararin, lutein, isradipine, lomoxicam, docetaxel, flurbiprofen, ciprofloxacin, furosemide, clopidogrel, dutasteride, amprenavir, saquinavir, calcitriol, valproic acid, isotretinoin, dronabinol, clofazimine, bexarotene, doxecalciferol, sirolimus, dutasteride, tipranavir, paricalcitol, topotecan, loratadine, nintedanib, calcifediol, and any combination thereof.

5. The lipid particle composition of claim 4, wherein the one or more poorly soluble active ingredients is selected from the group consisting of fenofibrate and cannabidiol.

6. The lipid particle composition of any one of claims 1-5, wherein the one or more lipids is selected from the group consisting of triglycerides, fatty acids, fluorinated lipids, neutral fats, phosphatides, oils, glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, and steroids.

7. The lipid particle composition of claim 6 wherein the one or more lipids comprises a triglyceride.

8. The lipid particle composition of claim 6 or 7, wherein the triglyceride is derived from one or both of glycerol and fatty acids.

9. The lipid particle composition of any one of claims 6-8, wherein the triglyceride comprises glyceryl tripalmitate.

10. The lipid particle composition of any one of claims 1-9, wherein the one or more lipids comprises a monoglyceride.

11. The lipid particle composition of claim 10, wherein the monoglyceride is selected from the group consisting of monolaurin, glyceryl monostearate, and glycerol hydroxy stearate.

12. The lipid particle composition of claim 11, wherein monoglyceride comprises glyceryl monostearate.

13. The lipid particle composition of any one of claims 1-12, wherein the one or more lipids comprises a diglyceride.

14. The lipid particle composition of any one of claims 1-13, wherein the one or more HLB modifying agents is selected from the group consisting of a surfactant, and an emulsifier.

15. The lipid particle composition of claim 14 comprising the surfactant, wherein the surfactant is selected from the group consisting of sodium lauryl sulphate, monooleate, monolaurate, monopalmitate, monostearate, an ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer®, Kolliphor EL, Tween™, and Gelucires™.

16. The lipid particle composition of claim 14 or 15 comprising the emulsifier, wherein the emulsifier is selected from the group consisting of egg lecithin, soy lecithin, and sodium lauryl sulphate.

17. The lipid particle composition of any one of claims 1-16, wherein the one or more poorly soluble active ingredients comprises at most about 30% by weight of the lipid particle composition.

18. The lipid particle composition of any one of claims 1-17, wherein the one or more HLB modifying agents comprises at most about 40% by weight of the lipid particle composition.

19. The lipid particle composition of any one of claims 1-18, wherein the one or more lipids comprises at most about 80% by weight of the lipid particle composition.

20. A method of manufacturing the solid lipid particle composition of any one of claims 1-19, wherein the method comprises spray coagulating at least two of the one or more poorly soluble active ingredients, the one or more lipids, and the one or more Hydrophile-Lipophile Balance (HLB) modifying agents.

21. A method of manufacturing the solid lipid particle composition of any one of claims 1-19, wherein the method comprises film casting at least two of the one or more poorly soluble active ingredients, the one or more lipids, and the one or more Hydrophile-Lipophile Balance (HLB) modifying agents.

22. A method of manufacturing the solid lipid particle composition of any one of claims 1-19, wherein the method comprises hot melting at least two of the one or more poorly soluble active ingredients, the one or more lipids, and the one or more Hydrophile-Lipophile Balance (HLB) modifying agents.

23. The method of claim 22, wherein the hot melting comprises one or both of hot melt extrusion and hot melt granulation.

24. A cannabidiol lipid particle composition, comprising:
one or more cannabidiols;
one or more lipids; and/or
one or more Hydrophile-Lipophile Balance (HLB) modifying agents to improve a bioavailability of the one or more poorly soluble active ingredients within a subject.

25. The cannabidiol lipid particle composition of claim 24, wherein the one or more lipids and/or the one or more HLB modifying agents comprise a solid system, and wherein the solid system is easily processed and/or stable at a temperature ranging from between about 45° C. and about 65° C.

26. The cannabidiol lipid particle composition of claim 24 or 25, wherein the one or more lipids is selected from group consisting of triglycerides, fatty acids, fluorinated lipids, neutral fats, phosphatides, oils, glycolipids, surface-active agents, surfactants, fluorosurfactants, aliphatic alcohols, waxes, terpenes, triglycerides, diglycerides, monoglycerides, hydrogenated vegetable oils, glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, and steroids.

27. The cannabidiol lipid particle composition of claim 26, wherein the one or more lipids comprises a triglyceride.

28. The cannabidiol lipid particle composition of claim 26 or 27, wherein the triglyceride comprises glyceryl tripalmitate.

29. The cannabidiol lipid particle composition of any one of claims 24-28, wherein the one or more lipids comprises monoglyceride.

30. The cannabidiol lipid particle composition of claim 29, wherein the monoglyceride comprises glyceryl monostearate.

31. The cannabidiol lipid particle composition of any one of claims 24-30, wherein the one or more lipids comprises diglyceride.

32. The cannabidiol lipid particle composition of any one of claims 24-31, wherein the one or more HLB modifying agents is selected from the group consisting of a surfactant and an emulsifier.

33. The cannabidiol lipid particle composition of claim 32 comprising the surfactant, wherein the surfactant comprises Kolliphor EL.

34. The cannabidiol lipid particle composition of claim 32 or 33 comprising the emulsifier, wherein the emulsifier is selected from the group consisting of egg lecithin, soy lecithin, and sodium lauryl sulphate.

35. A cannabidiol lipid particle composition, comprising:
one or more cannabidiols;
a glyceryl monostearate (Imwitor 900K);
a glycerol tripalmitate (Dynasan 116), and
a polyethoxylated castor oil (Kolliphor EL).

36. The cannabidiol lipid particle composition of claim 35, wherein the one or more cannabidiols comprises about 20% by weight of the cannabidiol lipid particle composition.

37. The cannabidiol lipid particle composition of claim 35 or 36, wherein the glyceryl monostearate (Imwitor 900K) comprises about 22% by weight of the cannabidiol lipid particle composition.

38. The cannabidiol lipid particle composition of any one of claims 35-37, wherein the glycerol tripalmitate (Dynasan 116) comprises about 35% by weight of the cannabidiol lipid particle composition.

39. The cannabidiol lipid particle composition of any one of claims 35-38, wherein the polyethoxylated castor oil (Kolliphor EL) comprises about 11% by weight of the cannabidiol lipid particle composition.

40. The cannabidiol lipid particle composition of any one of claims 35-39, further comprising lecithin.

41. The cannabidiol lipid particle composition of claim 40, wherein the lecithin comprises about 12% by weight of the cannabidiol lipid particle composition.

42. The cannabidiol lipid particle composition of claim 35, wherein the one or more cannabidiols comprises about 10% by weight of the cannabidiol lipid particle composition.

43. The cannabidiol lipid particle composition of claim 35 or 42, wherein the glyceryl monostearate (Imwitor 900K) comprises about 25% by weight of the cannabidiol lipid particle composition.

44. The cannabidiol lipid particle composition of any one of claims 35, 42, and 43, wherein the glycerol tripalmitate (Dynasan 116) comprises about 39% by weight of the cannabidiol lipid particle composition.

45. The cannabidiol lipid particle composition of any one of claims 35, and 42-44, wherein the polyethoxylated castor oil (Kolliphor EL) comprises about 13% by weight of the cannabidiol lipid particle composition.

46. The cannabidiol lipid particle composition of any one of claims 35, and 42-45, further comprising lecithin.

47. The cannabidiol lipid particle composition of claim 46, wherein the lecithin comprises about 13% by weight of the cannabidiol lipid particle composition.

48. The cannabidiol lipid particle composition of claim 35, wherein the one or more cannabidiols comprises about 5% by weight of the cannabidiol lipid particle composition.

49. The cannabidiol lipid particle composition of claim 35 or 48, wherein the glyceryl monostearate (Imwitor 900K) comprises about 27% by weight of the cannabidiol lipid particle composition.

50. The cannabidiol lipid particle composition of any one of claims 35, 48, and 49, wherein the glycerol tripalmitate (Dynasan 116) comprises about 41% by weight of the cannabidiol lipid particle composition.

51. The cannabidiol lipid particle composition of any one of claims 35, and 48-50, wherein the polyethoxylated castor oil (Kolliphor EL) comprises about 13% by weight of the cannabidiol lipid particle composition.

52. The cannabidiol lipid particle composition of any one of claims 35, and 48-51, further comprising lecithin.

53. The cannabidiol lipid particle composition of claim 52, wherein the lecithin comprises about 14% by weight of the cannabidiol lipid particle composition.

54. A method of manufacturing a cannabidiol lipid particle composition using spray coagulation, the method comprising:
(a) co-melting Imwitor 900K, Dynasan 116, and Kolliphor EL, thereby producing a melt;
(b) adding lecithin to the melt;
(c) adding a cannabidiol (CBD) to the melt;
(d) co-melting the melt with the lecithin and the CBD, thereby preparing a mixture,
(e) transferring the mixture to a heated vessel;
(f) equilibrating the mixture with the heated vessel;
(g) spray coagulating the mixture through a spraying chamber to achieve individual droplet separation; and
(h) cooling of the droplets to form solid lipid particles.

55. The method of claim 54, further comprising collecting the solid lipid particles.

56. The method of claim 54 or claim 55, further comprising coating or co-melting the solid lipid particles with a polymer.

57. The method of claim 54 or claim 55, further comprising preparing a solid suspension of the solid lipid particles with a material having a high melting point.

58. A method of manufacturing a cannabidiol lipid particle composition using film casting, the method comprising:
(a) co-melting Imwitor 900K, Dynasan 116, and Kolliphor EL, thereby producing a melt;
(b) adding lecithin to the melt;
(c) adding cannabidiol (CBD) to the melt;
(d) co-melting the melt with the lecithin and the CBD, thereby preparing a mixture;
(e) film casting the mixture, the film casting comprising pouring the mixture and cooling the mixture until hardened, thereby producing a solid lipid mixture.

59. The method of claim 58, further comprising grinding the solid lipid mixture into a lipid powder.

60. The method of claim 58 or 59, further comprising coating or co-melting the lipid powder with a polymer.

61. The method of claim 58 or 59, further comprising preparing a solid suspension of the lipid powder with a material having a high melting point.

62. A method of preparing a cannabidiol sachet or a stick pack, the method comprising:
(a) mixing solid lipid pellets of cannabidiol with a glidants;
(b) adding a sweetener, thereby producing a mixture; and
(c) packaging the mixture in a sachet or a stick pack.

63. A method of preparing a cannabidiol sachet or a stick pack, the method comprising:
(a) mixing solid lipid pellets of cannabidiol with a magnesium stearate;
(b) adding a fruit flavour and/or a sucralose, thereby producing a mixture; and
(c) packaging the mixture in a sachet or a stick pack.

64. A method of preparing a cannabidiol sachet or a stick pack, the method comprising:
(a) adding a carrier system comprising Mannogem EZ or Pharmasperse 415 with solid lipid particles of cannabidiol, thereby producing a first mixture;
(b) adding a magnesium stearate to the first mixture, thereby producing a second mixture;
(c) adding a fruit flavour and/or a sucralose to the second mixture; and
(d) packaging the mixture in a sachet or a stick pack.

65. A method of preparing a cannabidiol tablet composition, the method comprising:
(a) mixing the cannabidiol lipid particle composition of any one of claims 24-61 with at least one excipient or lubricant, thereby producing a blend; and
(b) compressing the blend into a tablet.

66. A method of preparing a cannabidiol tablet composition, the method comprising:
(a) mixing the cannabidiol lipid particle composition of any one of claims 24-61 with one or both of mannitol and magnesium stearate, thereby producing a blend; and
(b) compressing the blend into a tablet.

67. A solid dosage form for oral delivery comprising:
(a) an active ingredient; and
(b) a solid triglyceride;
wherein said active ingredient and solid triglyceride are spray congealed to form a bead.

68. A solid dosage form for oral delivery comprising:
(a) an active ingredient;
(b) a liquid triglyceride; and
(c) a silica,
wherein said active ingredient, liquid triglyceride, and silica are mixed to form a homogenous mixture.

69. The solid dosage form of claim 67 or 68, wherein the active ingredient is lipophilic.

70. The solid dosage form of claim 69, wherein the active ingredient is fenofibrate.

71. The solid dosage form of 67, wherein the solid triglyceride is selected from the group consisting of DYNASAN® 116, DYNASAN® 118, STEROTEX® GTP, STEROTEX® NF, STEROTEX® K, hydrogenated castor oil, and cocoa butter.

72. The solid dosage form of claim 68, wherein the liquid triglyceride is selected from the group consisting of sesame oil, olive oil, palm oil, cottonseed oil, corn oil, rapeseed oil, and safflower oil.

73. The solid dosage form of claim 68, wherein the silica is selected from the group consisting of SYLOID® XDP 3150, SYLOID® XDP 3050, ZEOPHARM™ 5191, and ZEOPHARM™ 600.

74. The solid dosage form of claim 67 or 68, wherein the active ingredient is present in an amount of about 5 mg/ml to about 100 mg/ml.

75. The solid dosage form of claim 74, wherein the active ingredient is present in an amount of about 10 mg/ml to about 75 mg/ml.

76. The solid dosage form of claim 75, wherein the active ingredient is present in an amount of about 50 mg/ml.

77. The solid dosage form of claim 75, wherein the active ingredient is present in an amount of about 70 mg/ml.

78. The solid dosage form of claim 67, further wherein said bead is combined with one or more other excipients to form a solid dosage form.

79. The solid dosage form of claim 68, further wherein said mixture is combined with one or more other excipients to form a solid dosage form.
